(19) United States
(12) Patent Application Publication
(10) Pub. No.: US 2024/0319349 A1
(43) Pub. Date: Sep. 26, 2024

(54) ULTRASONIC DIAGNOSTIC APPARATUS, ULTRASONIC DIAGNOSTIC METHOD AND NON-TRANSITORY COMPUTER READABLE MEDIUM

(71) Applicant: Canon Medical Systems Corporation, Otawara-shi (JP)

(72) Inventor: Akihiro KAKEE, Nasushiobara (JP)

(73) Assignee: Canon Medical Systems Corporation, Otawara-shi (JP)

(21) Appl. No.: 18/610,342

(22) Filed: Mar. 20, 2024

(30) Foreign Application Priority Data
Mar. 23, 2023 (JP) 2023-046794

(51) Int. Cl. G01S 7/52; A61B 8/00

(52) U.S. Cl. CPC G01S 7/5202; A61B 8/54

(57) ABSTRACT

According to one embodiment, an ultrasonic diagnostic apparatus includes processing circuitry. The processing circuitry computes depths of field of each of scan lines based on an ultrasonic image. The processing circuitry computes a corrected transmission voltage that makes the depths of field of the scan lines substantially uniform. The processing circuitry executes control to transmit an ultrasonic beam based on the corrected transmission voltage.

### CROSS-REFERENCE TO RELATED APPLICATION

This application is based upon and claims the benefit of priority from Japanese Patent Application No. 2023-046794, filed Mar. 23, 2023, the entire contents of which are incorporated herein by reference.

### FIELD

Embodiments described herein relate generally to an ultrasonic diagnostic apparatus, an ultrasonic diagnostic method and a non-transitory computer readable medium.

### BACKGROUND

In order to improve a viewing width of an ultrasonic image, there is a scanning method in which an angle of a scan line at an edge of a field of view is increased to acquire an ultrasonic image at a wide viewing angle. Although a viewing width is increased according to this scanning method, an angle of incidence of an ultrasonic beam at an edge of a field of view with respect to a living body is also increased, resulting in a trade-off problem in that sensitivity is degraded due to restriction of an element factor.

To address this problem, there is a method of improving the sensitivity by setting a transmission amplitude for each scan line according to an oscillation angle (angle of incidence) of an ultrasonic beam in an ultrasonic diagnostic apparatus capable of controlling an amplitude of a transmission drive voltage for each scan line. However, the magnitude of an echo signal varies according to not only an oscillation angle of an ultrasonic beam but also a manner of contact between an ultrasonic probe and a body surface, attenuation in a living body, etc., making it difficult to uniquely determine the setting of a transmission amplitude.

### BRIEF DESCRIPTION OF THE DRAWINGS

FIG. 1 is a block diagram showing an example of a configuration of an ultrasonic diagnostic apparatus according to an embodiment.

FIG. 2 is a flowchart for explaining an operation of the ultrasonic diagnostic apparatus according to the embodiment.

FIG. 3 is a diagram showing an example of setting a unit region according to the embodiment.

FIG. 4 is a graph showing a first example of a correspondence relationship between a depth of field of a scan line and a signal-to-noise ratio (SNR).

FIG. 5 is a graph showing a second example of a correspondence relationship between a depth of field of a scan line and an SNR.

FIG. 6 is a diagram showing an example of an ultrasonic image acquired by the ultrasonic diagnostic apparatus according to the embodiment.

### DETAILED DESCRIPTION

In general, according to one embodiment, an ultrasonic diagnostic apparatus includes processing circuitry. The processing circuitry computes depths of field of each of scan lines based on an ultrasonic image. The processing circuitry computes a corrected transmission voltage that makes the depths of field of the scan lines substantially uniform. The processing circuitry executes control to transmit an ultrasonic beam based on the corrected transmission voltage.

Hereinafter, an ultrasonic diagnostic apparatus, an ultrasonic diagnostic method and a non-transitory computer readable medium according to an embodiment will be described with reference to the accompanying drawings. In the embodiment described below, elements assigned with the same reference symbols are assumed to perform the same operations, and redundant descriptions thereof will be omitted as appropriate.

A process according to the embodiment described below is assumed to be applied to a case where a scanning method is performed in which an angle of a scan line at an edge of a field of view is increased to acquire an ultrasonic image at a wide viewing angle; however, the embodiment is not limited thereto. For example, a process according to the embodiment can be likewise applied to a scanning method in which there is a region where sensitivity is degraded in an imaging field of view, such as a scan line at an edge due to linear scanning.

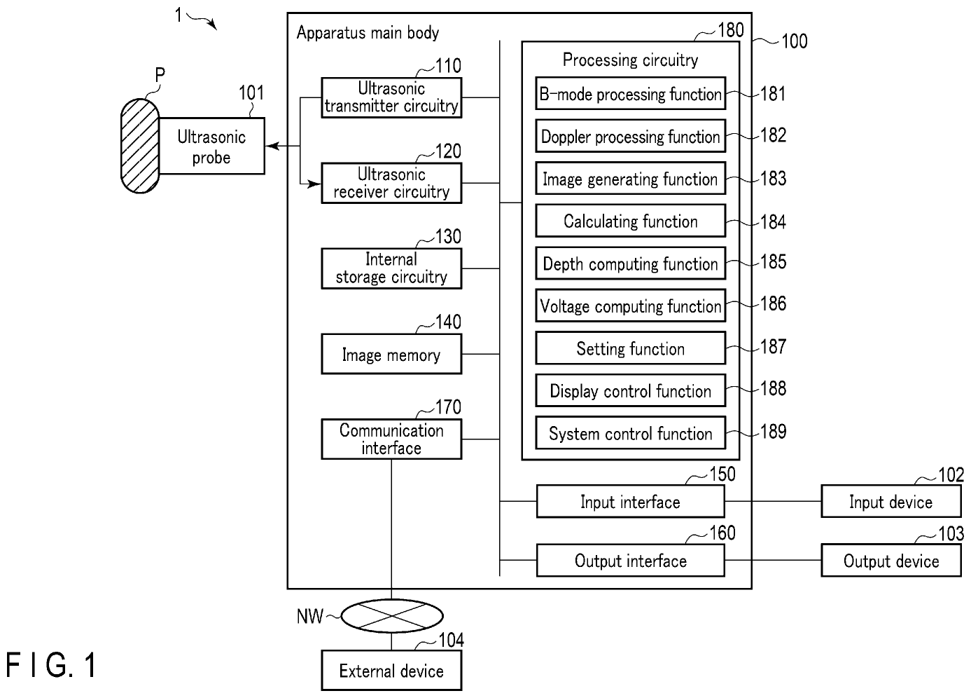

FIG. 1 is a diagram showing an example of a configuration of an ultrasonic diagnostic apparatus according to an embodiment. The ultrasonic diagnostic apparatus 1 shown in FIG. 1 includes an apparatus main body 100 and an ultrasonic probe 101. The apparatus main body 100 is connected to an input device 102 and an output device 103. The apparatus main body 100 is also connected to an external device 104 via a network NW. The external device 104 is, for example, a server equipped with picture archiving and communication systems (PACS) and a workstation capable of executing post processing.

The ultrasonic probe 101 executes ultrasonic scanning of a scan region inside a living body P, which is a subject, under the control of, for example, the apparatus main body 100. The ultrasonic probe 101 includes, for example, an acoustic lens, one or more matching layers, a plurality of vibrators (piezoelectric elements), a backing material, etc. The acoustic lens is formed of, for example, silicone rubber, and converges ultrasonic beams. The one or more matching layers perform impedance matching between the plurality of vibrators and the living body. The backing material prevents propagation of ultrasonic waves backward in a radial direction from the plurality of vibrators. The ultrasonic probe 101 is, for example, a one-dimensional array linear probe in which a plurality of vibrators are arranged along a predetermined direction. The ultrasonic probe 101 is detachably connected to the apparatus main body 100. The ultrasonic probe 101 may be provided with a button which is pressed when an offset process, an operation of freezing an ultrasonic image (i.e., freeze operation), etc., are performed.

The plurality of vibrators generate ultrasonic waves based on a drive signal supplied from ultrasonic transmitter circuitry 110 (described later) that is included in the apparatus main body 100. Ultrasonic waves are thereby transmitted from the ultrasonic probe 101 to the living body P. When the ultrasonic waves are transmitted from the ultrasonic probe 101 to the living body P, the transmitted ultrasonic waves are sequentially reflected on an acoustic impedance discontinuous surface of a body tissue of the living body P, and are received as echo signals by the plurality of piezoelectric vibrators. The amplitude of the received echo signals depends on a difference in acoustic impedance on the discontinuous surface to which the ultrasonic waves are reflected. If a transmitted ultrasonic pulse is reflected by a bloodstream or a surface of the cardiac wall or the like that is in motion, the frequency of the reflected echo signals is shifted due to the Doppler effect according to the moving object's velocity component in the direction of ultrasonic transmission. The ultrasonic probe 101 receives the reflected echo signals from the living body P, and converts the reflected echo signals into electric signals.

FIG. 1 illustrates a connection relationship between a single ultrasonic probe 101 and the apparatus main body 100. However, a plurality of ultrasonic probes can be connected to the apparatus main body 100. Which of the connected ultrasonic probes is to be used for the ultrasonic scanning can be selected freely through, for example, a software button on a touch panel described later.

The apparatus main body 100 generates an ultrasonic image based on the echo signal received by the ultrasonic probe 101. The apparatus main body 100 have ultrasonic transmitter circuitry 110, ultrasonic receiver circuitry 120, internal storage circuitry 130, an image memory 140, an input interface 150, an output interface 160, a communication interface 170, and processing circuitry 180.

The ultrasonic transmitter circuitry 110 is a processor that supplies a drive signal to the ultrasonic probe 101. The ultrasonic transmitter circuitry 110 is realized by, for example, a trigger generation circuit, a delay circuit, a pulser circuit, etc. The trigger generation circuit generates a rate pulse for forming ultrasonic waves for transmission repeatedly and at a predetermined rate frequency. The delay circuit gives a delay time for each piezoelectric vibrator to each rate pulse generated by the trigger generation circuit. This delay time is needed to converge the ultrasonic waves generated from the ultrasonic probe into a beam and determine the transmission directivity. The pulser circuit applies a drive signal (drive pulse) to a plurality of ultrasonic vibrators provided in the ultrasonic probe 101 at the timing based on the rate pulse. The transmission direction from the surfaces of the piezoelectric vibrators can be freely adjusted by varying the delay time given to each rate pulse by the delay circuit.

The ultrasonic transmitter circuitry 110 can freely change the output intensity of the ultrasonic waves through the drive signal. In the ultrasonic diagnostic apparatus, an influence of the attenuation of the ultrasonic waves in the living body P can be reduced by increasing the output intensity. The ultrasonic diagnostic apparatus can acquire an echo signal having a large signal-to-noise ratio (SNR) at the time of reception by reducing the influence of the attenuation of the ultrasonic waves.

In general, when an ultrasonic wave is propagated inside the living body P, the strength of the vibration of the ultrasonic wave (also referred to as “acoustic power”) corresponding to the output intensity is attenuated. The attenuation of the acoustic power is caused by absorption, scattering, reflection, etc. The degree of attenuation of the acoustic power depends on the frequency of the ultrasonic waves and the distance of the ultrasonic waves in the radial direction. For example, the degree of attenuation is increased by increasing the frequency of the ultrasonic waves. Also, the degree of attenuation is increased as the distance of the ultrasonic wave in the radiation direction becomes longer.

The ultrasonic receiver circuitry 120 is a processor that performs various kinds of processing on the received echo signals received by the ultrasonic probe 101 and generates received signals. The ultrasonic receiver circuitry 120 generates received signals for the echo signals of the ultrasonic waves obtained by the ultrasonic probe 101. Specifically, the ultrasonic receiver circuitry 120 is realized by, for example, a preamplifier, an A/D converter, a demodulator, a beamformer (adder), etc. The preamplifier performs gain correction by amplifying, for each channel, the echo signals received by the ultrasonic probe 101. The A/D converter converts the gain-corrected echo signals into digital signals. The demodulator demodulates the digital signals. The beam former, for example, gives the demodulated digital signals a delay time needed to determine the reception directivity, and adds a plurality of digital signals that have been given the delay time. The addition processing performed by the beam former generates received signals in which a reflection component from the direction corresponding to the reception directivity is emphasized. The received signals may also be referred to as IQ signals. In addition, the ultrasonic receiver circuitry 120 may store the received signals (IQ signals) in the internal storage circuitry 130 (described later), or output the received signals (IQ signals) to the external device 104 via the communication interface 170.

The internal storage circuitry 130 includes, for example, a processor-readable storage medium, such as a magnetic storage medium, an optical storage medium, or a semiconductor memory. The internal storage circuitry 130 stores therein programs, various types of data and the like for implementing ultrasonic transmission and reception. The programs and various types of data may be pre-stored in, for example, the internal storage circuitry 130. Alternatively, the programs and various types of data may be, for example, stored and distributed in a non-transitory storage medium, and read from the non-transitory storage medium to be installed in the internal storage circuitry 130. In accordance with an operation that is input via the input interface 150, the internal storage circuitry 130 stores B-mode image data, contrast image data, image data relating to a blood flow image, and the like that are generated by the processing circuitry 180. The internal storage circuitry 130 can also transfer the stored image data to the external device 104 or the like via the communication interface 170. The internal storage circuitry 130 may store the received signals (IQ signals) generated by the ultrasonic receiver circuitry 120, or transfer the received signals (IQ signals) to the external device 104 or the like via the communication interface 170.

The internal storage circuitry 130 may be a drive device or the like which reads and writes various kinds of information to and from a portable storage medium such as a CD drive, a DVD drive, and a flash memory. The internal storage circuitry 130 can also write the stored data into a portable storage medium, and store the data in the external device 104 via the portable storage medium.

The image memory 140 includes, for example, a processor-readable storage medium, such as a magnetic storage medium, an optical storage medium, or a semiconductor memory. The image memory 140 stores image data corresponding to multiple frames immediately preceding a freeze operation that is input via the input interface 150. The image data stored in the image memory 140 is, for example, continuously displayed (cine-displayed).

The internal storage circuitry 130 and the image memory 140 need not necessarily be realized by independent storage devices. The internal storage circuitry 130 and the image memory 140 may be realized by a single storage device. The internal storage circuitry 130 and the image memory 140 may each be realized by a plurality of storage devices.

The input interface 150 receives various instructions from an operator via the input device 102. The input device 102 is, for example, a mouse, a keyboard, a panel switch, a slider switch, a trackball, a rotary encoder, an operation panel, or a touch command screen (TCS). The input interface 150 is connected to the processing circuitry 180 via, for example, a bus, thereby converting an operation command that is input by the operator into an electric signal and outputting the electric signal to the processing circuitry 180. The input interface 150 is not limited to a component that is connected to physical operational components such as a mouse and a keyboard. Examples of the input interface also include circuitry that receives an electric signal corresponding to an operational command input from an external input device provided separately from the ultrasonic diagnostic apparatus 1 and outputs the electric signal to the processing circuitry 180.

The output interface 160 is, for example, an interface for outputting an electric signal from the processing circuitry 180 to the output device 103. The output device 103 is any display such as a liquid crystal display, an organic EL display, an LED display, a plasma display, or a CRT display. The output device 103 may be a touch-panel display that also serves as the input device 102. The output device 103 may further include a speaker that outputs voice in addition to a display. The output interface 160 is connected to the processing circuitry 180, for example, via a bus, and outputs the electric signal from the processing circuitry 180 to the output device 103.

The communication interface 170 is connected to the external device 104, for example, via the network NW to perform data communication with the external device 104.

The processing circuitry 180 is, for example, a processor acting as a nerve center of the ultrasonic diagnostic apparatus 1. The processing circuitry 180 executes a program stored in the internal storage circuitry 130, thereby implementing a function corresponding to the program. The processing circuitry 180 has, for example, a B-mode processing function 181, a Doppler processing function 182, an image generating function 183, a calculation function 184, a depth computing function 185, a voltage computing function 186, a setting function 187, a display control function 188, and a system control function 189.

The B-mode processing function 181 is a function to generate B-mode data based on a received signal from the ultrasonic receiver circuitry 120. With the B-mode processing function 181, the processing circuitry 180 performs, for example, envelope detection processing, logarithmic compression processing, and the like on the signal received from the ultrasonic receiver circuitry 120 to generate data (B-mode data) that expresses a signal intensity by brightness. The generated B-mode data is stored in a RAW data memory (not shown) as B-mode RAW data on a two-dimensional ultrasonic scan line (raster).

The Doppler processing function 182 is a function to analyze the frequency of the signal received from the ultrasonic receiver circuitry 120 and thereby generate data (Doppler information) of an extraction of Doppler effect-based motion information of a moving object present in a region of interest (ROI) set in a scan region. The generated Doppler information is stored in a RAW data memory (not shown) as Doppler RAW data (also referred to as “Doppler data”) on a two-dimensional ultrasonic scan line.

Specifically, with the Doppler processing function 182, the processing circuitry 180 estimates, at each sampling point, an average velocity, an average variance value, an average power value, etc., for example, as motion information of a moving object, and generates Doppler data showing the estimated motion information. The moving object is, for example, a bloodstream, tissue such as the cardiac wall, a contrast agent, etc. With the Doppler processing function 182, the processing circuitry 180 according to the embodiment estimates, at each sampling point, an average bloodstream velocity, a variance value of a bloodstream velocity, a power value of a bloodstream signal, etc., as motion information of a bloodstream (bloodstream information), and generates Doppler data showing the estimated bloodstream information.

Also, with the Doppler processing function 182, the processing circuitry 180 can perform a color Doppler method also referred to as a color flow mapping (CFM) method. In the CFM method, transmission and reception of ultrasonic waves on multiple scan lines are performed multiple times. In the CFM method, a moving target indicator (MTI) filter is applied to data columns in the same position, for example, whereby signals (clutter signals) originating from static tissue or slow-moving tissue are suppressed and signals originating from a blood flow are extracted. In the CFM method, the extracted blood flow signals are used to estimate blood flow information such as blood flow rate, blood flow dispersion, and blood flow power. With the image generating function 183 described later, a distribution of the estimated blood flow information is generated, for example, as ultrasonic image data (color Doppler image data) that is displayed two-dimensionally in color. Hereinafter, the mode of the ultrasonic diagnostic apparatus that adopts the color Doppler method will be referred to as a “blood flow imaging mode”. Color display refers to displaying a distribution of the blood flow information in accordance with a predetermined color code, and includes gray-scale color display.

There are various types of blood flow imaging modes depending on desired clinical information. In general, there is a blood flow imaging mode for displaying velocity that allows for visualization of a blood flow direction or an average blood flow rate, and a blood flow imaging mode for displaying power that allows for visualization of blood flow signal power.

The blood flow imaging mode for displaying velocity is a mode of displaying color corresponding to the Doppler shift frequency based on a blood flow direction or an average blood flow rate. For example, the blood flow imaging mode for displaying velocity represents, as flow directions, an approaching flow with a red-based color and a receding flow with a blue-based color, thereby representing the difference in the velocity between the approaching flow and the receding flow by the difference in the hue. The blood flow imaging mode for displaying velocity may also be called a “color Doppler mode” or a “color Doppler imaging (CDI) mode”.

The blood flow imaging mode for displaying power is a mode of representing blood flow signal power by, for example, a red-based color phase, brightness of the color, or a change in color saturation. The blood flow imaging mode for displaying power may also be called a “power Doppler (PD) mode”. Since the blood flow imaging mode for displaying power can represent the blood flow at a high sensitivity, as compared to the blood flow imaging mode for displaying velocity, the blood flow imaging mode for displaying power may be referred to as a high-sensitivity blood flow imaging mode.

The image generating function 183 is a function to generate B-mode image data based on the data generated by the B-mode processing function 181. With the image generating function 183, the processing circuitry 180, for example, converts (scan-converts) a scan line signal sequence of ultrasonic scanning into a scan line signal sequence in a video format representatively used by a television, etc., and generates image data for display (display image data). Specifically, the processing circuitry 180 generates two-dimensional B-mode image data (also referred to as “ultrasonic image data”) constituted by pixels by executing RAW-pixel conversion, such as coordinate conversion according to the mode of the ultrasonic scanning performed by the ultrasonic probe 101, on B-mode RAW data stored in the RAW data memory. In other words, with the image generating function 183, the processing circuitry 180 generates multiple ultrasonic images (medical images) corresponding to respective consecutive frames through ultrasonic transmission and reception.

The image generating function 183 also has a function to generate Doppler image data based on the data generated by the Doppler processing function 182. For example, the image generating function 183 generates Doppler image data of visualized blood flow information by executing RAW-pixel conversion on the Doppler RAW data stored in the RAW data memory. The Doppler image data is average velocity image data, dispersion image data, power image data, or combined image data thereof. The processing circuitry 180 generates, as the Doppler image data, color Doppler image data showing colored blood flow information and Doppler image data showing a piece of blood flow information in waveform on a gray scale. The color Doppler image data is generated when the above-described blood flow imaging modes are executed.

The calculation function 184 calculates an average value and a variance value of pixel values in a unit region set in an ultrasonic image and computes an SNR from the average value.

The depth computing function 185 computes a depth of field of each scan line based on the ultrasonic image.

The voltage computing function 186 computes a corrected transmission voltage that makes the depths of field of the scan lines substantially uniform.

The setting function 187 sets a new pulse repetition frequency (PRF) for each scan line according to the corrected transmission voltage.

The display control function 188 is a function of causing a display as the output device 103 to display images based on various kinds of ultrasonic image data generated by the image generating function 183. Specifically, with the display control function 188, the processing circuitry 180, for example, controls the display of an image that is based on the B-mode image data, the Doppler image data, or image data including both that is generated by the image generating function 183.

More specifically, with the display control function 188, the processing circuitry 180, for example, converts (scan-converts) a scan line signal sequence of ultrasonic scanning into a scan line signal sequence in a video format representatively used by a television, etc., and generates display image data. The processing circuitry 180 may also perform various kinds of processing, such as corrections of dynamic range, brightness, contrast, and a γ-curve and RGB conversion, on the display image data. The processing circuitry 180 may also add supplementary information, such as textual information of various parameters, a scale, or a body mark, to the display image data. The processing circuitry 180 may also generate a user interface (graphical user interface: GUI) for an operator to input various commands through the input device, and cause the display to display the GUI.

The system control function 189 is a function to perform overall control of the operations of the ultrasonic diagnostic apparatus 1. For example, the system control function 189 controls the ultrasonic transmitter circuitry 110, etc., so as to transmit an ultrasonic beam based on the corrected transmission voltage and the new PRF.

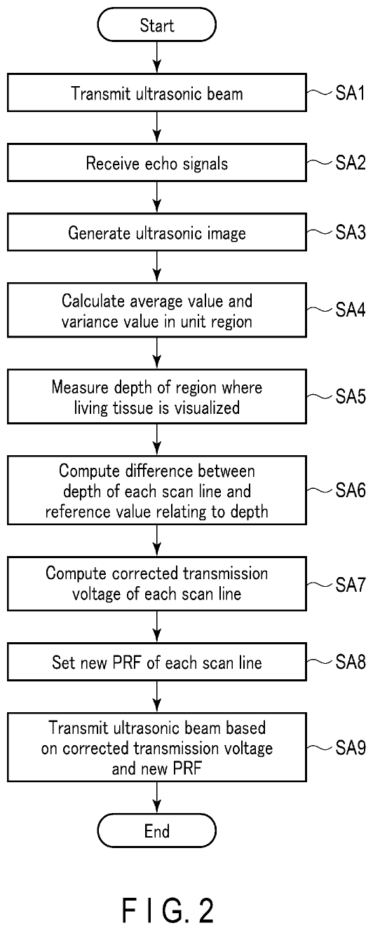

Next, an example of an operation of the ultrasonic diagnostic apparatus according to the embodiment, that is, a process of correcting a transmission voltage, will be described with reference to the flowchart of FIG. 2. If the correcting process according to the embodiment is performed while a process of acquiring an ultrasonic image is already being performed, steps SA1 to SA3 of acquiring an original ultrasonic image may be omitted.

In step SA1, the ultrasonic transmitter circuitry 110 transmits an ultrasonic beam to a living body P, which is a subject.

In step SA2, the ultrasonic receiver circuitry 120 receives an echo signal from the living body P.

In step SA3, by implementing the B-mode processing function 181, the processing circuitry 180 performs, for example, the signal processing described above on the echo signal and generates an ultrasonic image.

In step SA4, by implementing the calculation function 184, the processing circuitry 180 sets a unit region defined by m sample (s) in a depth direction and n scan line (s) (n raster (s)) in a scanning direction in an ultrasonic image. Herein, both “m” and “n” are an integer equal to or greater than 1. With the calculation function 184, the processing circuitry 180 calculates, for each unit region, an average value of pixels corresponding to the sample (s) included in the unit region by performing an averaging process such as LPF (Low-Pass Filter), and further calculates a variance value for the pixels included in the unit region.

By implementing the calculation function 184, the processing circuitry 180 specifies, based on the average value and the variance value, a unit region in which a living tissue is visualized. Specifically, by implementing the calculation function 184, the processing circuitry 180 computes, as an SNR (also referred to as “sensitivity”), a difference between the average value computed in step SA3 and an average value of noise components (such as white noise). Also, by setting a range of a variance value according to each living tissue (such as a soft tissue, a structure, etc.), it is possible to determine that a living tissue is visualized in a unit region where the SNR is equal to or above a threshold and the variance value belongs to a predetermined range of values.

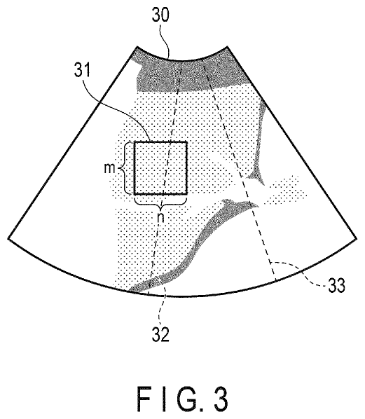

In step SA5, by implementing the depth computing function 185, the processing circuitry 180 computes a depth of field (penetration) of each scan line based on the unit region in which it is determined that a living tissue is visualized. For example, by implementing the depth computing function 185, the processing circuitry 180 may compute a maximum depth in the unit region in which SNR of an image is equal to or below a threshold and a living tissue is visualized, as the depths of field of the scan lines included in the unit region. By implementing the depth computing function 185, the processing circuitry 180 may approximate a distribution of SNR in the focus of an ultrasonic beam and an area beyond it with a straight line or a curved line, and compute a depth that provides an SNR equal to or below a predetermined SNR. A specific example of computing a depth of field of a scan line will be described later with reference to FIGS. 3 to 5.

In step SA6, by implementing the depth computing function 185, the processing circuitry 180 computes, based on a target depth of field set in advance or an average value or a maximum value of a depth of field of each scan line as a reference value, a difference between the maximum depth of each scan line and the reference value.

In step SA7, by implementing the voltage computing function 186, the processing circuitry 180 computes, based on the difference computed in step SA6 and various parameters relating to an ultrasonic beam, such as a received center frequency, an attenuation coefficient, and a correction coefficient, a corrected transmission voltage, which is a transmission voltage computed so that the difference is equal to or below a threshold and is not beyond a controlled value (upper limit) of acoustic power. The controlled value of acoustic power is, for example, an upper limit of thermal energy generated by ultrasonic absorption of a living tissue. By implementing the voltage computing function 186, the processing circuitry 180 may compute a corrected transmission voltage not based on a received center frequency and a correction coefficient that are constant values but based on a received center frequency and a correction coefficient that are adaptively varied for each scan line. The processing circuitry 180 may also compute a corrected transmission voltage such that an average value of a transmission amplitude relating to a transmission voltage does not differ from that for transmitting an ultrasonic beam in step SA1.

In step SA8, by implementing the setting function 187, the processing circuitry 180 sets a new PRF according to the corrected transmission voltage. Specifically, the processing circuitry 180, for example, sets a new PRF by updating the original repetition frequency of each scan line according to a ratio of the corrected transmission voltage to the former transmission voltage for acquiring an ultrasonic image in step SA1. This is performed for the purpose of preventing a heat-generating temperature from excessively rising in each scan line. By implementing the setting function 187, the processing circuitry 180 may set a new PRF for the corrected transmission voltage of each scan line, for example, according to the following formula (1):

New PRF=min(original PRF×√(corrected transmission voltage/original transmission voltage),highest PRF)

Herein, the “highest PRF” refers to a preset maximum update rate that is needed to secure a depth of field of an ultrasonic image. Thus, either one of the following, whichever is smaller, may be set as a new PRF, as shown in the above formula (1): a value obtained by multiplying the square root of the ratio of the corrected transmission voltage to the former transmission voltage by the former PRF; or a preset highest PRF.

If a target depth of field can be set smaller than the original depth of field (the depth of field set when an ultrasonic image is acquired in step SA1), the corrected transmission voltage can also be set lower than the original transmission voltage. In this case, the amount of heat generated by a living body is also small, thus allowing for setting of a PRF higher than the original one.

In step SA9, by implementing the system control function 189, the processing circuitry 180 controls the ultrasonic transmitter circuitry 110 and the ultrasonic probe 101 based on the corrected transmission voltage and the new PRF that is set in step SA8, and transmits an ultrasonic beam to the living body P for each scan line.

Next, an example of setting a unit region according to the embodiment will be described with reference to FIG. 3.

FIG. 3 shows an example in which a rectangular m (sample)×n (raster) unit region 31 is set in an ultrasonic image 30. In the ultrasonic image 30, a portion showing a pattern indicates that a living tissue is visualized, and a blank portion indicates that a living tissue is not visualized. The shape of the unit region 31 is not limited to a rectangular shape, and may be any shape such as a circular shape, a sector shape, or the like. Unit regions are sequentially set in the entire ultrasonic image 30, and whether or not a living tissue is visualized therein is determined. By implementing the depth computing function 185, the processing circuitry 180 may determine a maximum depth in the unit region 31 in which an SNR is less than a threshold to be a value of the depth of field of the scan line included in the unit region 31.

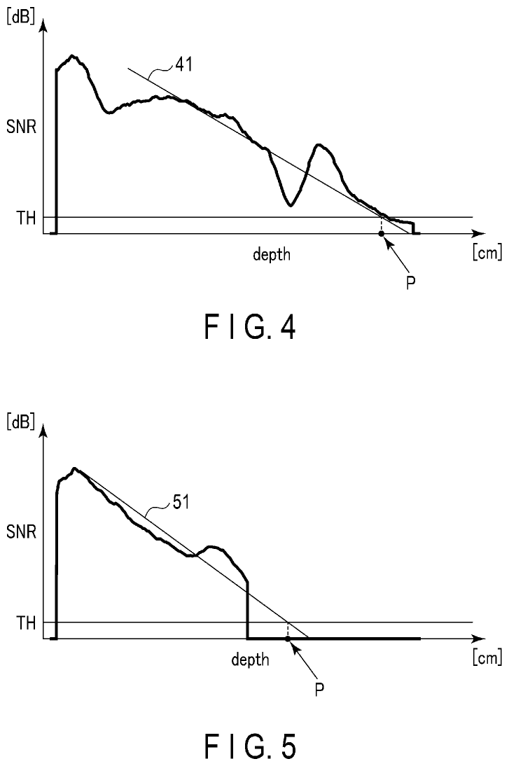

Next, a first example of a correspondence relationship between a depth of field of a scan line and an SNR will be explained with reference to the graph shown in FIG. 4.

FIG. 4 is a graph connecting two-dimensional plots that shows the correspondence between the depth of field and the SNR in connection with the scan line 32 corresponding to the center of the ultrasonic image 30 shown in FIG. 3. The horizontal axis shows a depth of a unit region (scan line), and the vertical axis shows an SNR. Since a living tissue is visualized almost entirely along the depth direction of the scan line 32, the SNR and the depth of field are in a proportional relationship. That is, as the depth in a direction toward the inside of the body increases, the SNR value gradually decreases.

In this instance, by implementing the depth computing function 185, the processing circuitry 180, for example, calculates an approximate straight line 41 by a linear regression method, and sets a depth at the intersection between the approximate straight line 41 and an SNR threshold TH as a depth of field P [cm]. A second- or higher-order approximate curve may be used instead of the approximate straight line 41, and an intersection between the SNR graph itself and the threshold may be computed.

Next, a second example of a correspondence relationship between a depth of field of a scan line and an SNR will be explained with reference to the graph shown in FIG. 5.

FIG. 5 is a graph connecting two-dimensional plots that shows the correspondence between the depth of field and the SNR in connection with the scan line 33 corresponding to the end side of the ultrasonic image 30 shown in FIG. 3. With regard to the scan line 33, although there is a living tissue on the upper side of the ultrasonic image 30 (the body surface side of the living body P), no biological tissue is visualized on the lower side of the ultrasonic image 30 (the inner side of the living body P). In this instance, the value of the SNR is zero in an area at 8 [cm] or deeper.

In this instance, by implementing the depth computing function 185, the processing circuitry 180 performs linear regression on the graph and extrapolates an approximate straight line 51. By implementing the depth computing function 185, the processing circuitry 180 can set a depth at the intersection between the approximate straight line and an SNR threshold TH as a depth of field P [cm]. In this manner, a corrected transmission voltage that presupposes visualization of an image up to a predetermined depth of field can be computed even for a scan line of an ultrasonic beam that is transmitted to a portion where there is no biological tissue.

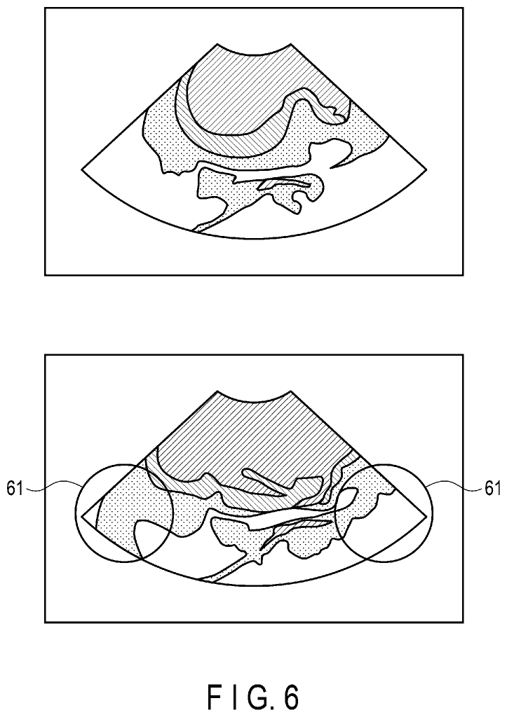

Next, an example of an ultrasonic image acquired by the ultrasonic diagnostic apparatus 1 according to the embodiment will be explained with reference to FIG. 6.

The upper figure of FIG. 6 is an ultrasonic image acquired by a conventional method, and the lower figure of FIG. 6 is an ultrasonic image acquired by the ultrasonic diagnostic apparatus 1 according to the embodiment. The ultrasonic images shown in FIG. 6 are superimposed on an ordinary B-mode image to be displayed by dividing the SNR value in multiple stages. In the ultrasonic image shown in the upper figure of FIG. 6, which is acquired by a conventional method, a constant transmission voltage is set for all the scan lines irrespective of the positions of the scan lines; thus, the transmission voltage is scarce on the end side of the ultrasonic image, resulting in an image having a low SNR.

On the other hand, according to the ultrasonic image shown in the lower figure of FIG. 6, which the ultrasonic diagnostic apparatus 1 of the embodiment acquires by performing a voltage correction process, an ultrasonic beam is transmitted with a corrected transmission voltage in connection with the scan line in the region 61 on the end side of the ultrasonic image; thus, the SNR (sensitivity) can be maintained at a high level in the region 61, and the brightness of the image can also be improved.

According to the embodiment described above, the presence or absence of visualization of a living tissue is determined for each unit region, and a depth of field is computed for each scan line. A corrected transmission voltage that makes the depths of field substantially uniform among the scan lines is computed, and an ultrasonic beam is transmitted based on the corrected transmission voltage. In addition, a new PRF is set according to the corrected transmission voltage, an ultrasonic beam is transmitted in each scan line based on the new PRF with a transmission amplitude that is based on the corrected transmission voltage, and an ultrasonic image is generated. Thus, even in a scanning method that sets a wide viewing angle, for example, it is possible to maintain the sensitivity (increase the SNR) for a scan line at an end where the brightness tends to be low, enabling provision of an ultrasonic image with a wide field of view and high image quality. As a result, the accuracy of diagnostic imaging can be improved.

The term “processor” used in the above description means, for example, circuitry such as a central processing unit (CPU), a graphics processing unit (GPU), an application-specific integrated circuit (ASIC), or a programmable logic device (e.g., a simple programmable logic device (SPLD), a complex programmable logic device (CPLD), or a field-programmable gate array (FPGA)). If the processor is, for example, a CPU, the processor implements the functions by reading and executing programs stored in storage circuitry. On the other hand, if the processor is an ASIC, for example, its functions are directly incorporated into the circuitry of the processor as logic circuitry, instead of a program being stored in the storage circuitry. Each processor of the present embodiment is not limited to be configured as single circuitry; multiple sets of independent circuitry may be integrated into a single processor that implements its functions. Furthermore, the functions may be implemented by a single processor into which multiple components shown in the drawings are incorporated.

In addition, the functions described in the above embodiment may be implemented by installing programs for executing the processing in a computer, such as a workstation, and expanding the programs in a memory. The programs that can cause the computer to execute the processing can be stored in a storage medium, such as a magnetic disk (a hard disk, etc.), an optical disk (CD-ROM, DVD, etc.), or a semiconductor memory and distributed through it.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising processing circuitry configured to:
compute depths of field of each of scan lines based on an ultrasonic image;
compute a corrected transmission voltage that makes the depths of field of the scan lines substantially uniform; and
execute control to transmit an ultrasonic beam based on the corrected transmission voltage.

2. The ultrasonic diagnostic apparatus according to claim 1, wherein the processing circuitry is further configured to set a new pulse repetition frequency according to the corrected transmission voltage for each of the scan lines.

3. The ultrasonic diagnostic apparatus according to claim 2, wherein the processing circuitry is configured to set, as the new pulse repetition frequency, either one of (a) a value obtained by multiplying a square root of a ratio of the corrected transmission voltage to a transmission voltage for acquiring the ultrasonic image by a pulse repetition frequency for acquiring the ultrasonic image, or (b) a preset maximum pulse repetition frequency, whichever is smaller.

4. The ultrasonic diagnostic apparatus according to claim 1, wherein the processing circuitry is configured to compute a difference between the depth of field and a reference value, and compute the corrected transmission voltage such that the difference is equal to or below a threshold and is not beyond an upper limit of acoustic power transmitted to a subject.

5. The ultrasonic diagnostic apparatus according to claim 1, wherein the processing circuitry is configured to compute a maximum depth in a unit region set in the ultrasonic image as the depth of field of the scan line included in the unit region, the unit region has an image signal-to-noise ratio being equal to or below a first threshold and a living tissue being visualized.

6. The ultrasonic diagnostic apparatus according to claim 5, wherein the processing circuitry is configured to:
calculate an average value and a variance value of pixel values in the unit region set in the ultrasonic image;
compute the signal-to-noise ratio from the average value; and
determine that the living tissue is visualized in the unit region if the signal-to-noise ratio is equal to or above a second threshold and the variance value belongs to a predetermined range.

7. The ultrasonic diagnostic apparatus according to claim 1, wherein the processing circuitry is configured to compute, as the depth of field, an intersection between an approximate straight line or an approximate curve and a first threshold of a signal-to-noise ratio in an image of a unit region set in the ultrasonic image, the approximate straight line or the approximate curve being extrapolated with respect to a graph represented by the signal-to-noise ratio and a depth corresponding to the unit region.

8. An ultrasonic diagnostic method comprising:
computing depths of field of each of scan lines based on an ultrasonic image;
computing a corrected transmission voltage that makes the depths of field of the scan lines substantially uniform; and
executing control to transmit an ultrasonic beam based on the corrected transmission voltage.

9. A non-transitory computer readable medium including computer executable instructions, wherein the instructions, when executed by a processor, cause the processor to perform a method comprising:
computing depths of field of each of scan lines based on an ultrasonic image;
computing a corrected transmission voltage that makes the depths of field of the scan lines substantially uniform; and
executing control to transmit an ultrasonic beam based on the corrected transmission voltage.
